(19) United States
(12) Patent Application Publication
(10) Pub. No.: US 2024/0354599 A1
(43) Pub. Date: Oct. 24, 2024

(54) METHOD FOR DETERMINING PROBABILITY OF A KIDNEY STONE IN A SUBJECT BEING A URIC-ACID STONE

(71) Applicants: KAOHSIUNG MEDICAL UNIVERSITY, Kaohsiung City (TW); National Sun Yat-Sen University, Kaohsiung City (TW)

(72) Inventors: Hao-Wei Chen, Kaohsiung City (TW); Chung-Yao Kao, Kaohsiung City (TW); Jung-Ting Lee, Kaohsiung City (TW); Yu-Chen Chen, Kaohsiung City (TW)

(73) Assignees: KAOHSIUNG MEDICAL UNIVERSITY, Kaohsiung City (TW); National Sun Yat-Sen University, Kaohsiung City (TW)

(21) Appl. No.: 18/304,594

(22) Filed: Apr. 21, 2023

(51) Int. Cl. G06N 5/022; A61B 5/20; G16H 50/30

(52) U.S. Cl. CPC G06N 5/022; A61B 5/201; G16H 50/30

(57) ABSTRACT

A method for determining a probability of a kidney stone in a subject being a uric-acid (UA) stone includes steps of: establishing, by using a machine learning algorithm, a prediction model based on a plurality of training data sets that are related to a plurality of patients, each of the plurality of training data sets at least including an estimated glomerular filtration rate (eGFR) and a value of urine pH; and feeding an input variable set into the prediction model so as to obtain the probability of the kidney stone in the subject being a UA stone. The input variable set is related to the subject and including an eGFR and a value of urine pH of the subject.

### FIELD

The disclosure relates to a method for determining a probability of a kidney stone in a subject being a uric-acid (UA) stone, and more particularly to a machine-learning-based method for determining a probability of a kidney stone in a subject being a UA stone.

### BACKGROUND

Nephrolithiasis is a crystallopathy where a patient has a kidney stone developing in his/her urinary tract. According to composition of the kidney stone, nephrolithiasis could be roughly classified as uric-acid (UA) stone disease or non-UA stone disease. Patients with UA stone disease account for 10% to 15% of patients with nephrolithiasis. Many studies have indicated that UA stone disease is related to features of metabolic syndrome and nutrient partitioning disorders (e.g., diabetes mellitus and obesity). Unlike non-UA stone disease, most UA stone diseases do not require surgery to treat.

Dual-energy computed tomography (CT) is considered an accurate test that can be conducted before treatment for nephrolithiasis to differentiate UA stone disease from non-UA stone disease. However, equipment of dual-energy CT is prohibitively expensive for most clinics around the world, and thus is not widely available. In addition, concerns about safety arise due to the radiation exposure associated with using dual-energy CT.

### SUMMARY

Therefore, an object of the disclosure is to provide a method for determining a probability of a kidney stone in a subject being a uric-acid (UA) stone that can alleviate at least one of the drawbacks of the prior art.

According to the disclosure, the method includes steps of: establishing, by using a machine learning algorithm, a prediction model based on a plurality of training data sets that are related to a plurality of patients, each of the plurality of training data sets at least including an estimated glomerular filtration rate (eGFR) and a value of urine pH; and feeding an input variable set into the prediction model so as to obtain the probability of the kidney stone in the subject being a UA stone, the input variable set being related to the subject and including an eGFR and a value of urine pH of the subject.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a prediction system for determining a probability of a kidney stone in a subject being a uric-acid (UA) stone according to an embodiment of the disclosure.

FIG. 2 is a block diagram illustrating the prediction system according to another embodiment of the disclosure.

FIG. 3 is a block diagram illustrating the prediction system according to still another embodiment of the disclosure.

FIG. 4 is a flow chart illustrating a method for determining a probability of a kidney stone in a subject being a UA stone according to an embodiment of the disclosure.

FIGS. 5 and 6 are receiver operating characteristic (ROC) curves cooperatively showing performance of the method according to a first embodiment of the disclosure.

FIGS. 7 and 8 are ROC curves cooperatively showing performance of the method according to a second embodiment of the disclosure.

FIGS. 9 and 10 are ROC curves cooperatively showing performance of the method according to a third embodiment of the disclosure.

FIGS. 11 and 12 are ROC curves cooperatively showing performance of the method according to a fourth embodiment of the disclosure.

FIGS. 13 and 14 are ROC curves cooperatively showing performance of the method according to a fifth embodiment of the disclosure.

FIGS. 15 and 16 are ROC curves cooperatively showing performance of the method according to a sixth embodiment of the disclosure.

FIGS. 17 and 18 are ROC curves cooperatively showing performance of the method according to a seventh embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

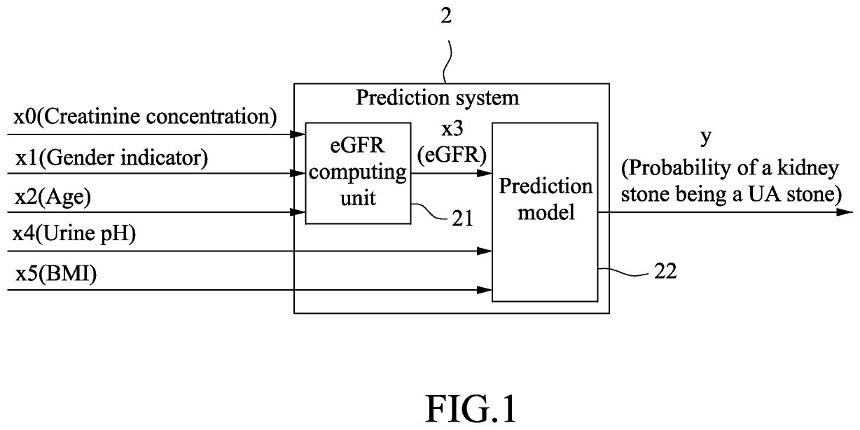

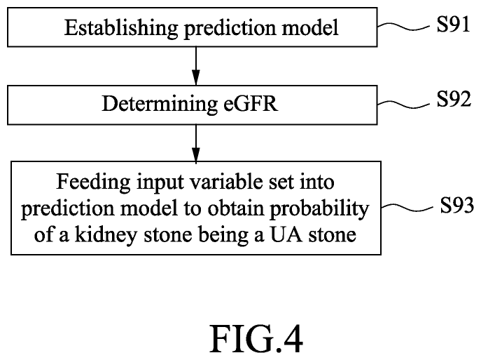

Referring to FIG. 4, an embodiment of a method for determining a probability of a kidney stone in a subject being a uric-acid (UA) stone according to the disclosure is illustrated. The subject is a person who has been diagnosed with nephrolithiasis (i.e., kidney stone disease). The method is to be implemented by a prediction system 2 as exemplarily shown in FIGS. 1, 2 and 3. The prediction system 2 may be implemented by a desktop computer, a laptop computer, a notebook computer, a tablet computer, a processor, a central processing unit (CPU), a microprocessor, a micro control unit (MCU), a system on a chip (SoC), or any circuit configurable/programmable in a software manner and/or hardware manner to implement functionalities discussed in this disclosure.

The method at least includes steps S91 and S93 delineated below.

In step S91, the prediction system 2 establishes, by using a machine learning algorithm, a prediction model 22 based on a plurality of training data sets that are related to a plurality of patients. Each of the plurality of training data sets at least includes an estimated glomerular filtration rate (eGFR) and a value of urine pH of a patient. The prediction model 22 is mathematically expressed as

$$y = \frac{1}{1 + {e}^{f ( x )}} ,$$

where y represents output of the prediction model 22 (i.e., a probability of a kidney stone being a UA stone) and has a value ranging from zero to one, x represents input to the prediction model 22 and is in the form of a vector, and ƒ(x) is a function of the input to the prediction model 22 and will be further explained later. When the output of the prediction model 22 has a value of one, it means that the probability of the kidney stone being a UA stone is 100%; when the output of the prediction model 22 has a value of zero, it means that the probability of the kidney stone being a UA stone is 0%. However, representation of output of the prediction model 22 is not limited to the disclosure herein and may vary in other embodiments. The prediction model 22 is built using the Python programming language and built-in libraries of Python, and is trained by applying a built-in optimization engine of Python with a weighted binary cross-entropy function serving as the objective function. Model parameters of the prediction model 22 are trained based on the training data sets. Since implementing a prediction model by using a machine learning algorithm has been well known to one skilled in the relevant art (e.g., reference may be made to an MIT Press book “Deep Learning” authored by Goodfellow, Bengio and Courville), detailed explanation of the same is omitted herein for the sake of brevity.

In step S93, the prediction system 2 feeds an input variable set into the prediction model 22 so as to obtain the probability of the kidney stone in the subject being a UA stone. The input variable set is related to the subject and at least includes an eGFR and a value of urine pH of the subject.

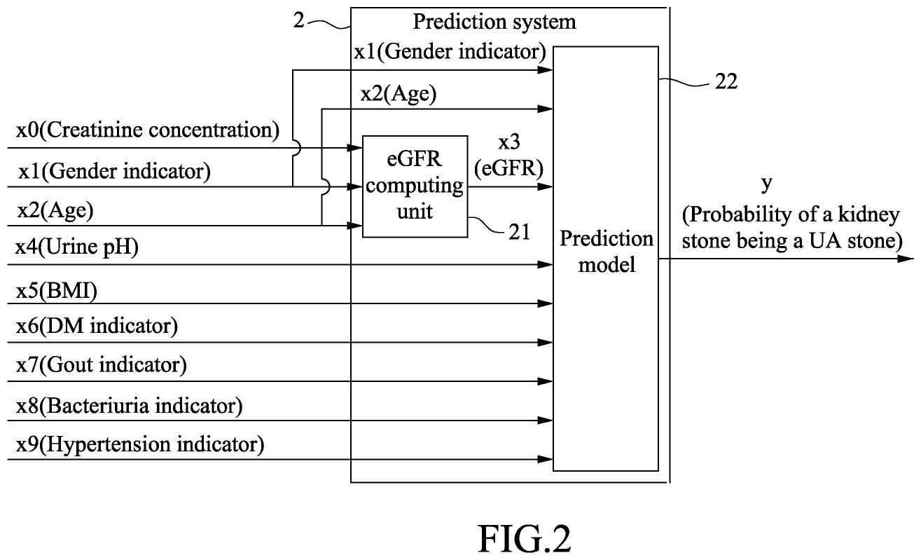

In some embodiments, each of the plurality of training data sets further includes an age of a patient, and a gender indicator that indicates gender of the patient (e.g., one for female and zero for male), and a creatinine concentration that is related to the patient. The prediction system 2 includes an eGFR computing unit 21. In such embodiments, the input variable set further includes an age of the subject, a gender indicator that indicates gender of the subject (e.g., one for female and zero for male), and a creatinine concentration that is related to the subject. However, in some embodiments (e.g., the embodiments shown in FIGS. 1 and 2), the eGFR is not directly received by the prediction system 2 but is calculated by the eGFR computing unit 21 based on the age, the gender indicator and the creatinine concentration, and then is fed into the prediction model 22 together with other variables as the input variable set. The eGFR computing unit 21 may be implemented by one of hardware, firmware, software, or any combination thereof. For example, the eGFR computing unit 21 may be implemented by software modules in a program, where the software modules contain codes and instructions to carry out specific functionalities, and can be called individually or together to fulfill functions described in this disclosure. The software modules may be embodied in: executable software as a set of logic instructions stored in a machine- or computer-readable storage medium of a memory such as random access memory (RAM), read only memory (ROM), programmable ROM (PROM), firmware, flash memory, etc.; configurable logic such as programmable logic arrays (PLAs), field programmable gate arrays (FPGAs), complex programmable logic devices (CPLDs), etc.; fixed-functionality logic hardware using circuit technology such as application specific integrated circuit (ASIC), complementary metal oxide semiconductor (CMOS), transistor-transistor logic (TTL) technology, etc.; or any combination thereof.

Accordingly, in these embodiments, prior to step S93, the method further includes step S92. In step S92, the eGFR computing unit 21 of the prediction system 2 calculates the eGFR based on the age, the gender indicator and the creatinine concentration. Specifically, the eGFR computing unit 21 calculates the eGFR by using the isotope dilution mass spectrometry traceable Modification of Diet in Renal Disease formula that is mathematically expressed as

$$eGFR [ mL / min / 1 \text{.73} {m}^{2} ] = 175 \times {( Scr )}^{- 1.154} \times {( Age )}^{- 0.203} \times {( 0.742 )}^{G} ,$$

where Scr represents the creatinine concentration, Age represents the age, and G represents the gender indicator which has a value of one indicating that the subject is a female, or a value of zero indicating that the subject is a male. For information about the isotope dilution mass spectrometry traceable Modification of Diet in Renal Disease formula, reference may be made to “A new equation to estimate glomerular filtration rate” authored by Levey, Stevens, Schmid, Zhang, Castro, Feldman, Kusek, Egger, Van Lente, Greene, et al. in Annals of Internal Medicine, 2009, and detailed explanation thereof is omitted herein for the sake of brevity.

In some embodiments where each of the plurality of training data sets includes the age of a patient, the gender indicator that indicates gender of the patient, a creatinine concentration that is related to the patient, or a combination thereof, the prediction system 2 may omit the eGFR computing unit 21, in which case the input variable set is directly received by the prediction system 2 (e.g., inputted by a user using an input device of the prediction system 2) and then fed into the prediction model 22.

In order to validate performance of the method according to the disclosure, data collected from two groups of patients who have been diagnosed with nephrolithiasis was used. A first group of patients (hereinafter also referred to as “Cohort A”) includes 1098 patients, and 146 patients thereamong (i.e., 13.3% of the patients of Cohort A) have UA stone disease. A second group of patients (hereinafter also referred to as “Cohort B”) includes 71 patients, 3 patients thereamong (i.e., 4.23% of the patients of Cohort B) have UA stone disease. It is worth to note that determination as to whether a patient of Cohort A or Cohort B has UA stone disease or not is made based on analysis of infrared spectroscopy performed on a kidney stone that is taken out from the patient by surgery. For Cohort A, data from sixty percent of the patients in Cohort A was used to serve as the training data sets for training the prediction model 22, and data from the remaining forty percent of the patients in Cohort A was used to serve as validation data sets. It is worth to note that the aforesaid division of the data from Cohort A into the training data sets and the validation data sets is devised such that the proportion of the patients whose data is used as the training data sets and who have UA stone disease to all the patients whose data is used as the training data sets is approximate to the proportion of the patients whose data is used as the validation data sets and who have UA stone disease to all the patients whose data is used as the validation data sets, so as to ensure that there is only a minor statistical difference between the training data sets and the validation data sets. For Cohort B, data related to all the patients in Cohort B was used to serve as validation data sets. Predictive power (i.e., accuracy of prediction) of the prediction model 22 applied to the validation data (which is collected from Cohort A and Cohort B) is evaluated by using receiver operating characteristic (ROC) analysis based on an area under curve (AUC) that is related to an ROC curve. Specifically, in ROC analysis, an optimal cutoff point on the ROC curve is determined based on Youden's index. Then, the optimal cutoff point is used to calculate sensitivity, specificity, positive predictive value (PPV), and negative predictive value (NPV). Since ROC analysis has been well known to one skilled in the relevant art, detailed explanation of the same is omitted herein for the sake of brevity.

In a first embodiment of the method, each of the plurality of training data sets includes the eGFR and the value of urine pH of a patient, the prediction system 2 includes the eGFR computing unit 21, and the input variable set includes the eGFR and the value of urine pH of the subject. Particularly, the prediction system 2 receives a variable x₀ that represents the creatinine concentration, a variable x₁ that represents the gender indicator, a variable x₂ that represents the age, and a variable x₄ that represents the value of urine pH. The eGFR computing unit 21 calculates, based on the variables x₀,x₁ and x₂, a variable x₃ that represents the eGFR, and then the variable x₃ thus calculated is fed into the prediction model 22 together with the variable x₄ as the input variable set. In response to receipt of the input variable set, the prediction model 22 obtains the probability of a kidney stone being a UA stone.

In the mathematical expression of the prediction model 22 according to the first embodiment, ƒ(x) is a function that is mathematically expressed as ƒ(x)=w₀+w₃(x)ƒ₃(x)+w₄(x)ƒ₄(x), where w₀ is a constant, each of w₃(x), w₄(x), ƒ₃(x) and ƒ₄(x) is a function, and w₀, w₃(x), w₄(x), ƒ₃(x) and ƒ₄(x) are determined by using a fully connected neural network. In particular, the function ƒ(x) can be simplified as ƒ(x)=w₀+w₃ƒ₃(x₃)+w₄ƒ₄(x₄), where each of w₀, w₃ and w₄ is a scalar trainable parameter, and each of ƒ₃(x₃) and ƒ₄(x₄) is a nonlinear function determined by using a fully connected two-layer neural network. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. Each of ƒ₃(x₃) and ƒ₄(x₄) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

where i is equal to 3 or 4, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{i,2} is a standard nonlinear activation function commonly used in building a neural network. In total, the function ƒ(x) contains Σᵢ₌₃⁴3mᵢ+5 number of trainable model parameters. In this embodiment, each of m₃ and ma is equal to 20, each of B_{3,1} and B_{4,1} is manually set as a zero vector, and the function ƒ(x) contains only 85 trainable model parameters, accordingly.

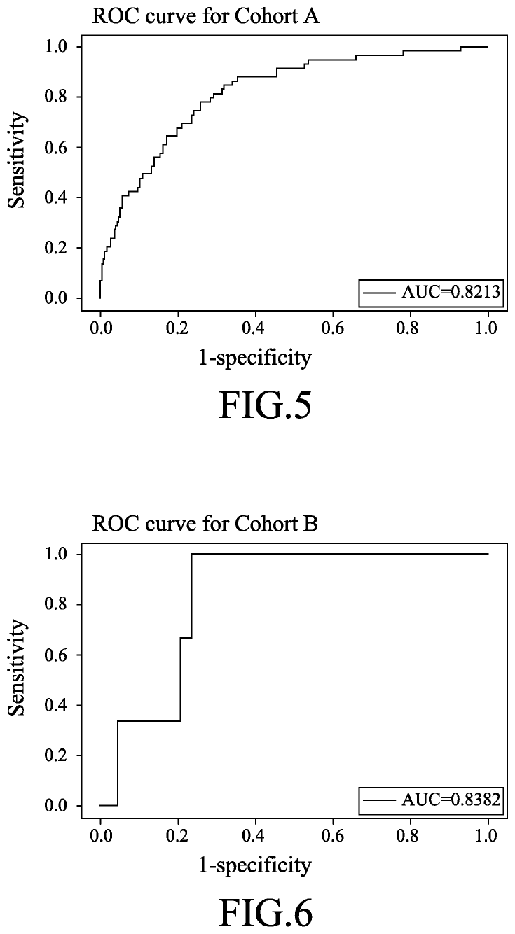

For the prediction model 22 according to the first embodiment, an ROC curve for the prediction model 22 fed with the validation data sets collected from Cohort A is illustrated in FIG. 5, and another ROC curve for the prediction model 22 fed with the validation data sets collected from Cohort B is illustrated in FIG. 6. An AUC related to the ROC curve in FIG. 5 is 0.8213, and an AUC related to said another ROC curve in FIG. 6 is 0.8382.

Referring to FIG. 1, in a second embodiment of the method, each of the plurality of training data sets includes the eGFR and the value of urine pH of a patient, and a body mass index (BMI) that is related to the patient. The prediction system 2 includes the eGFR computing unit 21, and the input variable set includes the eGFR, the value of urine pH and the body mass index (BMI) of the subject. Particularly, the prediction system 2 receives a variable x₀ that represents the creatinine concentration, a variable x₁ that represents the gender indicator, a variable x₂ that represents the age, a variable x₄ that represents the value of urine pH, and a variable x₅ that represents the BMI. The eGFR computing unit 21 calculates, based on the variables x₀,x₁ and x₂, a variable x₃ that represents the eGFR, and then the variable x₃ thus calculated is fed into the prediction model 22 together with the variables x₄ and x₅ as the input variable set. In response to receipt of the input variable set, the prediction model 22 obtains the probability of a kidney stone being a UA stone.

In the mathematical expression of the prediction model 22 according to the second embodiment, ƒ(x) is a function that is mathematically expressed as ƒ(x)=w₀+w₃(x)ƒ₃(x)+w₄(x)ƒ₄(x)+w₅(x)ƒ₅(x), where w₀ is a constant, each of w₃(x), w₄(x), w₅(x), ƒ₃(x), ƒ₄(x) and ƒ₅(x) is a function, and w₀, w₃(x), w₄(x), w₅(x), ƒ₃(x), ƒ₄(x) and ƒ₅(x) are determined by using a fully connected neural network. In particular, the function ƒ(x) can be simplified as ƒ(x)=w₀+w₃ƒ₃(x₃)+w₄ƒ₄(x₄)+w₅ƒ₅(x₅), where each of w₀, w₃, w₄ and w₅ is a scalar trainable parameter, and each of ƒ₃(x₃), ƒ₄(x₄) and ƒ₅(x₅) is a nonlinear function determined by using a fully connected two-layer neural network. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. Each of ƒ₃(x₃), ƒ₄(x₄) and ƒ₅(x₅) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

where i is equal to 3, 4 or 5, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{i,2} is a standard nonlinear activation function commonly used in building a neural network. In total, the function ƒ(x) contains Σᵢ₌₃⁵3mᵢ+7 number of trainable model parameters. In this embodiment, each of m₃, m₄ and m₅ is equal to 20, each of B_{3,1}, B_{4,1} and B_{5,1} is manually set as a zero vector, and the function ƒ(x) contains only 127 trainable model parameters, accordingly.

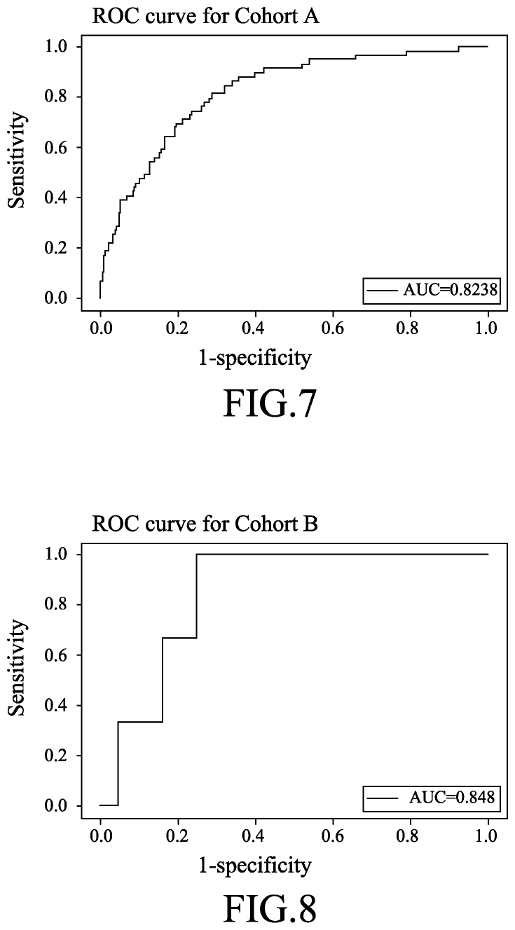

For the prediction model 22 according to the second embodiment, an ROC curve for the prediction model 22 fed with the validation data sets collected from Cohort A is illustrated in FIG. 7, and another ROC curve for the prediction model 22 fed with the validation data sets collected from Cohort B is illustrated in FIG. 8. An AUC related to the ROC curve in FIG. 7 is 0.8238, and an AUC related to said another ROC curve in FIG. 8 is 0.848.

In a third embodiment of the method, each of the plurality of training data sets includes the eGFR, the value of urine pH, the BMI and the age of a patient. The prediction system 2 includes the eGFR computing unit 21, and the input variable set includes the eGFR, the value of urine pH, the BMI and the age of the subject. Particularly, the prediction system 2 receives a variable x₀ that represents the creatinine concentration, a variable x₁ that represents the gender indicator, a variable x₂ that represents the age, a variable x₄ that represents the value of urine pH, and a variable x₅ that represents the BMI. The eGFR computing unit 21 calculates, based on the variables x₀,x₁ and x₂, a variable x₃ that represents the eGFR, and then the variable x₃ thus calculated is fed into the prediction model 22 together with the variables x₂,x₄ and x₅ as the input variable set. In response to receipt of the input variable set, the prediction model 22 obtains the probability of a kidney stone being a UA stone.

In the mathematical expression of the prediction model 22 according to the third embodiment, ƒ(x) is a function that is mathematically expressed as ƒ(x)=w₀+w₂(x)ƒ₂(x)+w₃(x)ƒ₃(x)+w₄(x)ƒ₄(x)+w₅(x)ƒ₅(x), where w₀ is a constant, each of w₂(x), w₃(x), w₄(x), w₅(x), ƒ₂(x), ƒ₃(x), ƒ₄(x) and ƒ₅(x) is a function, and w₀, w₂(x), w₃(x), w₄(x), w₅(x), ƒ₂(x), ƒ₃(x), ƒ₄(x) and ƒ₅(x) are determined by using a fully connected neural network. In particular, the function ƒ(x) can be simplified as ƒ(x)=w₀+w₂ƒ₂(x₂)+w₃ƒ₃(x₃)+w₄ƒ₄(x₄)+w₅ƒ₅(x₅), where each of w₀, w₂, w₃, w₄ and w₅ is a scalar trainable parameter, and each of ƒ₂(x₂), ƒ₃(x₃), ƒ₄(x₄) and ƒ₅(x₅) is a nonlinear function determined by using a fully connected two-layer neural network. For ƒ₂(x₂), a first layer of the fully connected two-layer neural network has a dimension m₂ that is an integer ranging from 1 to 100, and a second layer of the fully connected two-layer neural network has a dimension of 1. For ƒ₃(x₃), a first layer of the fully connected two-layer neural network has a dimension m₃ that is an integer ranging from 1 to 100, and a second layer of the fully connected two-layer neural network has a dimension of 1. For fa (x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. Each of ƒ₂(x₂), ƒ₃(x₃), ƒ₄(x₄) and ƒ₅(x₅) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

where i is equal to 2, 3, 4 or 5, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{i,2} is a standard nonlinear activation function commonly used in building a neural network. In total, the function ƒ(x) contains Σᵢ₌₂⁵3mᵢ+9 number of trainable model parameters. In this embodiment, each of m₂, m₃, m₄ and m₅ is equal to 20, each of B_{2,1}, B_{3,1}, B_{4,1} and B_{5,1} is manually set as a zero vector, and the function ƒ(x) contains only 169 trainable model parameters, accordingly.

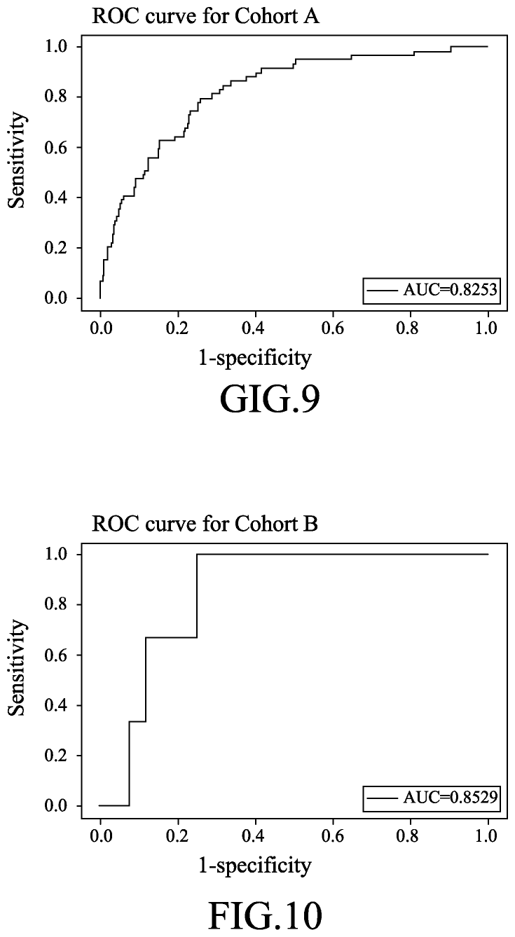

For the prediction model 22 according to the third embodiment, an ROC curve for the prediction model 22 fed with the validation data sets collected from Cohort A is illustrated in FIG. 9, and another ROC curve for the prediction model 22 fed with the validation data sets collected from Cohort B is illustrated in FIG. 10. An AUC related to the ROC curve in FIG. 9 is 0.8253, and an AUC related to said another ROC curve in FIG. 10 is 0.8529.

In a fourth embodiment of the method, each of the plurality of training data sets includes the eGFR, the value of urine pH, the BMI and the age of a patient, and the gender indicator indicating gender of the patient. The prediction system 2 includes the eGFR computing unit 21, and the input variable set includes the eGFR, the value of urine pH, the BMI and the age of the subject, and the gender indicator indicating gender of the subject. Particularly, the prediction system 2 receives a variable x₀ that represents the creatinine concentration, a variable x₁ that represents the gender indicator, a variable x₂ that represents the age, a variable x₄ that represents the value of urine pH, and a variable x₅ that represents the BMI. The eGFR computing unit 21 calculates, based on the variables x₀,x₁ and x₂, a variable x₃ that represents the eGFR, and then the variable x₃ thus calculated is fed into the prediction model 22 together with the variables x₁,x₂,x₄ and x₅ as the input variable set for the prediction model 22 to obtain the probability of a kidney stone being a UA stone.

In the mathematical expression of the prediction model 22, ƒ(x) is a function that is mathematically expressed as ƒ(x)=w₀+w₁(x)ƒ₁(x)+w₂(x)ƒ₂(x)+w₃(x)ƒ₃(x)+w₄(x)ƒ₄(x)+w₅(x)ƒ₅(x), where w₀ is a constant, each of w₁(x), w₂(x), w₃(x), w₄(x), w₅(x), ƒ₁(x), ƒ₂(x), ƒ₃(x), ƒ₄(x) and ƒ₅(x) is a function, and w₀, w₁(x), w₂(x), w₃(x), w₄(x), w₅(x), ƒ₁(x), ƒ₂(x), ƒ₃(x), ƒ₄(x) and ƒ₅(x) are determined by using a fully connected neural network. In particular, the function ƒ(x) can be simplified as ƒ(x)=w₀+w₁ƒ₁(x₁)+w₂ƒ₂(x₂)+w₃ƒ₃(x₃)+w₄ƒ₄(x₄)+w₅ƒ₅(x₅), where each of w₀, w₁, w₂, w₃, w₄ and w₅ is a scalar trainable parameter, and each of ƒ₁(x₁), ƒ₂(x₂), ƒ₃(x₃), ƒ₄(x₄) and ƒ₅(x₅) is a nonlinear function determined by using a fully connected two-layer neural network. For ƒ₁(x₁), a first layer of the fully connected two-layer neural network has a dimension m₁ that is an integer ranging from 1 to 100, and a second layer of the fully connected two-layer neural network has a dimension of 1. For ƒ₂(x₂), a first layer of the fully connected two-layer neural network has a dimension m₂ that is an integer ranging from 1 to 100, and a second layer of the fully connected two-layer neural network has a dimension of 1. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. Each of ƒ₁(x₁), ƒ₂(x₂), ƒ₃(x₃), ƒ₄(x₄) and ƒ₅(x₅) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

where i is equal to 1, 2, 3, 4 or 5, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{i,2} is a standard nonlinear activation function commonly used in building a neural network. In total, the function ƒ(x) contains Σᵢ₌₁⁵3mᵢ+11 number of trainable model parameters. In this embodiment, each of m₁, m₂, m₃, m₄ and m₅ is equal to 20, each of B_{1,1}, B_{2,1}, B_{3,1}, B_{4,1} and B_{5,1} is manually set as a zero vector, and the function ƒ(x) contains only 211 trainable model parameters, accordingly.

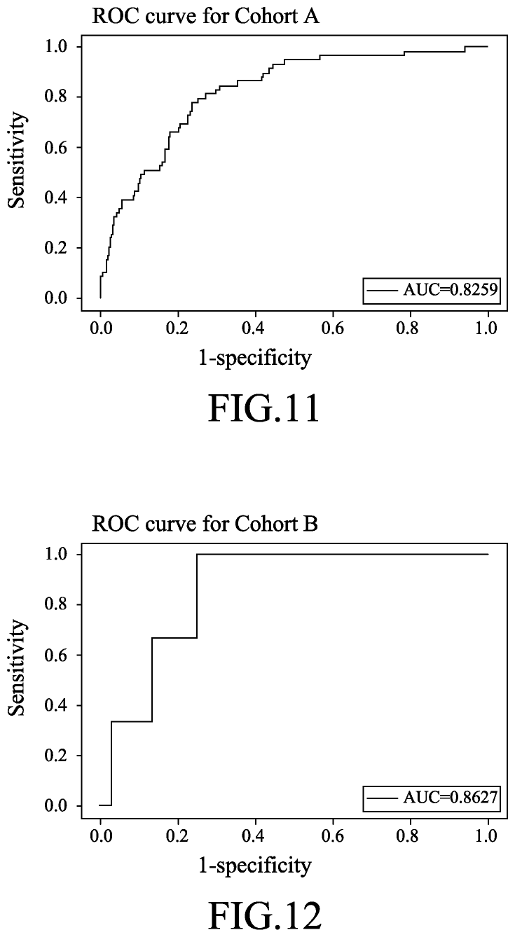

For the prediction model 22 according to the fourth embodiment, an ROC curve for the prediction model 22 fed with the validation data sets collected from Cohort A is illustrated in FIG. 11, and another ROC curve for the prediction model 22 fed with the validation data sets collected from Cohort B is illustrated in FIG. 12. An AUC related to the ROC curve in FIG. 11 is 0.8259, and an AUC related to said another ROC curve in FIG. 12 is 0.8627.

In a fifth embodiment of the method, each of the plurality of training data sets includes the eGFR, the value of urine pH, the BMI and the age of a patient, the gender indicator indicating gender of the patient, and a diabetes mellitus (DM) indicator indicating whether the patient was ever diagnosed with DM. For example, the DM indicator may have a value of one to indicate that the patient had once been diagnosed with DM, or a value of zero to indicate that the patient was never diagnosed with DM. The prediction system 2 includes the eGFR computing unit 21.

In the fifth embodiment, the input variable set includes the eGFR, the value of urine pH, the BMI and the age of the subject, the gender indicator indicating gender of the subject, and a DM indicator indicating whether the subject was ever diagnosed with DM. For example, the DM indicator may have a value of one to indicate that the subject had once been diagnosed with DM, or a value of zero to indicate that the subject was never diagnosed with DM. Particularly, the prediction system 2 receives a variable x₀ that represents the creatinine concentration, a variable x₁ that represents the gender indicator, a variable x₂ that represents the age, a variable x₄ that represents the value of urine pH, a variable x₅ that represents the BMI, and a variable x₆ that represents the DM indicator. The eGFR computing unit 21 calculates, based on the variables x₀,x₁ and x₂, a variable x₃ that represents the eGFR, and then the variable x₃ thus calculated is fed into the prediction model 22 together with the variables x₁,x₂,x₄,x₅ and x₆ as the input variable set for the prediction model 22 to obtain the probability of a kidney stone being a UA stone.

In the mathematical expression of the prediction model 22, ƒ(x) is a function that is mathematically expressed as ƒ(x)=w₀+w₁(x)ƒ₁(x)+w₂(x)ƒ₂(x)+w₃(x)ƒ₃(x)+w₄(x)ƒ₄(x)+w₅(x)ƒ₅(x)+w₆(x)ƒ₆(x), where w₀ is a constant, each of w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) is a function, and w₀, w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) are determined by using a fully connected neural network. In particular, the function ƒ(x) can be simplified as ƒ(x)=w₀+w₁ƒ₁(x₁)+w₂ƒ₂(x₂)+w₃ƒ₃(x₃)+w₄ƒ₄(x₄)+w₅ƒ₅(x₅)+w₆ƒ₆(x₆), where each of w₀, w₁, w₂, w₃, w₄, w₅ and w₆ is a scalar trainable parameter, and each of ƒ₁(x₁), ƒ₂(x₂), ƒ₃(x₃), ƒ₄(x₄), ƒ₅(x₅) and ƒ₆(x₆) is a nonlinear function determined by using a fully connected two-layer neural network. For ƒ₁(x₁), a first layer of the fully connected two-layer neural network has a dimension m₁ that is an integer ranging from 1 to 100, and a second layer of the fully connected two-layer neural network has a dimension of 1. For ƒ₂(x₂), a first layer of the fully connected two-layer neural network has a dimension m₂ that is an integer ranging from 1 to 100, and a second layer of the fully connected two-layer neural network has a dimension of 1. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. For ƒ₆(x₆), a first layer of the fully connected two-layer neural network has a dimension m₆ that is an integer ranging from 1 to 100, and a second layer of the fully connected two-layer neural network has a dimension of 1. Each of ƒ₁(x₁), ƒ₂(x₂), ƒ₃(x₃), ƒ₄(x₄), ƒ₅(x₅) and ƒ₆(x₆) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

where i is equal to 1, 2, 3, 4, 5 or 6, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{1,2} is a standard nonlinear activation function commonly used in building a neural network. In total, the function ƒ(x) contains Σᵢ₌₁⁶3mᵢ+13 number of trainable model parameters. In this embodiment, each of m₁, m₂, m₃, m₄, m₅ and m₆ is equal to 20, each of B_{1,1}, B_{2,1}, B_{3,1}, B_{4,1}, B_{5,1} and B_{6,1} is manually set as a zero vector, and the function ƒ(x) contains only 253 trainable model parameters, accordingly.

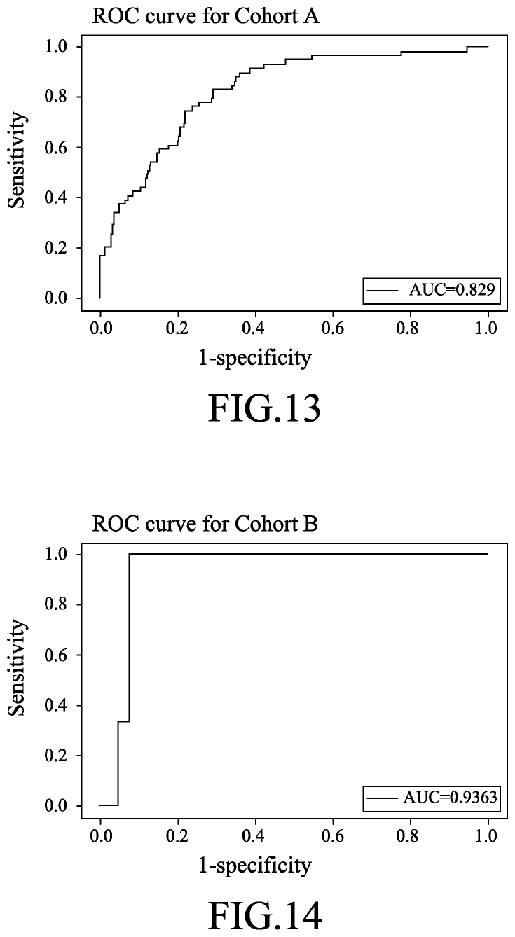

For the prediction model 22 according to the fifth embodiment, an ROC curve for the prediction model 22 fed with the validation data sets collected from Cohort A is illustrated in FIG. 13, and another ROC curve for the prediction model 22 fed with the validation data sets collected from Cohort B is illustrated in FIG. 14. An AUC related to the ROC curve in FIG. 13 is 0.829, and an AUC related to said another ROC curve in FIG. 14 is 0.9363.

In a sixth embodiment of the method, each of the plurality of training data sets includes the eGFR, the value of urine pH, the BMI and the age of a patient, the gender indicator indicating gender of the patient, the DM indicator indicating whether the patient was ever diagnosed with DM, a gout indicator indicating whether the patient was ever diagnosed with gout, and a bacteriuria indicator indicating whether the patient was ever diagnosed with bacteriuria. For example, the gout indicator may have a value of one to indicate that the patient had once been diagnosed with gout, or a value of zero to indicate that the patient was never diagnosed with gout. Similarly, the bacteriuria indicator may have a value of one to indicate that the patient had once been diagnosed with bacteriuria, or a value of zero to indicate that the patient was never diagnosed with bacteriuria. The prediction system 2 includes the eGFR computing unit 21.

In the sixth embodiment, the input variable set includes the eGFR, the value of urine pH, the BMI and the age of the subject, the gender indicator indicating gender of the subject, the DM indicator indicating whether the subject was ever diagnosed with DM, a gout indicator indicating whether the subject was ever diagnosed with gout, and a bacteriuria indicator indicating whether the subject was ever diagnosed with bacteriuria. For example, the gout indicator may have a value of one to indicate that the subject had once been diagnosed with gout, or a value of zero to indicate that the subject was never diagnosed with gout. Similarly, the bacteriuria indicator may have a value of one to indicate that the subject had once been diagnosed with bacteriuria, or a value of zero to indicate that the subject was never diagnosed with bacteriuria. Particularly, the prediction system 2 receives a variable x₀ that represents the creatinine concentration, a variable x₁ that represents the gender indicator, a variable x₂ that represents the age, a variable x₄ that represents the value of urine pH, a variable x₅ that represents the BMI, a variable x₆ that represents the DM indicator, a variable x₇ that represents the gout indicator and a variable x₈ that represents the bacteriuria indicator. The eGFR computing unit 21 calculates, based on the variables x₀,x₁ and x₂, a variable x₃ that represents the eGFR, and then the variable x₃ thus calculated is fed into the prediction model 22 together with the variables x₁,x₂,x₄,x₅,x₆,x₇ and x₈ as the input variable set for the prediction model 22 to obtain the probability of a kidney stone being a UA stone.

In the mathematical expression of the prediction model 22, ƒ(x) is a function that is mathematically expressed as ƒ(x)=w₀+w₁(x)ƒ₁(x)+w₂(x)ƒ₂(x)+w₃(x)ƒ₃(x)+w₄(x)ƒ₄(x)+w₅(x)ƒ₅(x)+w₆(x)ƒ₆(x), where w₀ is a constant, each of w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) is a function, and w₀, w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) are determined by using a fully connected neural network. In particular, the function ƒ(x) can be simplified as ƒ(x)=w₀+w₁ƒ₁(x₁)+w₂ƒ₂(x₂)+w₃ƒ₃(x₃)+w₄ƒ₄(x₄)+w₅ƒ₅(x₅)+w₆ƒ₆(x₆,x₇,x₈), where each of w₀, w₁, w₂, w₃, w₄, w₅ and w₆ is a scalar trainable parameter, and each of ƒ₁(x₁), ƒ₂(x₂), ƒ₃(x₃), ƒ₄(x₄), ƒ₅(x₅) and ƒ₆(x₆,x₇,x₈) is a nonlinear function determined by using a fully connected two-layer neural network. For ƒ₁(x₁), a first layer of the fully connected two-layer neural network has a dimension m₁ that is an integer ranging from 1 to 100, and a second layer of the fully connected two-layer neural network has a dimension of 1. For ƒ₂(x₂), a first layer of the fully connected two-layer neural network has a dimension m₂ that is an integer ranging from 1 to 100, and a second layer of the fully connected two-layer neural network has a dimension of 1. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. For ƒ₆(x₆,x₁,x₈), a first layer of the fully connected two-layer neural network has a dimension m₆ that is an integer ranging from 1 to 100, and a second layer of the fully connected two-layer neural network has a dimension of 1. Each of ƒ₁(x₁), ƒ₂(x₂), ƒ₃(x₃), ƒ₄(x₄) and ƒ₅(x₅) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

and
ƒ₆(x₆,x₇,x₈) is mathematically expressed as

$${f}_{6} ( {x}_{6} , {x}_{7} , {x}_{8} ) = {σ}_{6 , 2} ( {A}_{6 , 2} {σ}_{6 , 1} ( {A}_{6 , 1} {x}_{6} + {A}_{7 , 1} {x}_{7} + {A}_{8 , 1} {x}_{8} + {B}_{6 , 1} ) + {B}_{6 , 2} ) ,$$

where i is equal to 1, 2, 3, 4 or 5, each of A_{i,1} and B_{i,1} is an mᵢ-by-1 vector containing mᵢ number of trainable model parameters, A_{i,2} is a 1-by-mᵢ vector containing mᵢ number of trainable model parameters, B_{i,2} is a scalar trainable parameter, and each of σ_{i,1} and σ_{1,2} is a standard nonlinear activation function commonly used in building a neural network, each of A_{6,1}, A_{7,1}, A_{8,1} and B_{6,1} is an m₆-by-1 vector containing m₆ number of trainable model parameters, A_{6,2} is a 1-by-m₆ vector containing m₆ number of trainable model parameters, B_{6,2} is a scalar trainable parameter, and each of σ_{6,1} and σ_{6,2} is a standard nonlinear activation function commonly used in building a neural network. In total, the function ƒ(x) contains Σᵢ₌₁⁶3mᵢ+2m₆+13 number of trainable model parameters. In this embodiment, each of m₁, m₂, m₃, m₄, m₅ and m₆ is equal to 20, each of B_{1,1}, B_{2,1}, B_{3,1}, B_{4,1}, B_{5,1} and B_{6,1} is manually set as a zero vector, and the function ƒ(x) contains only 293 trainable model parameters, accordingly.

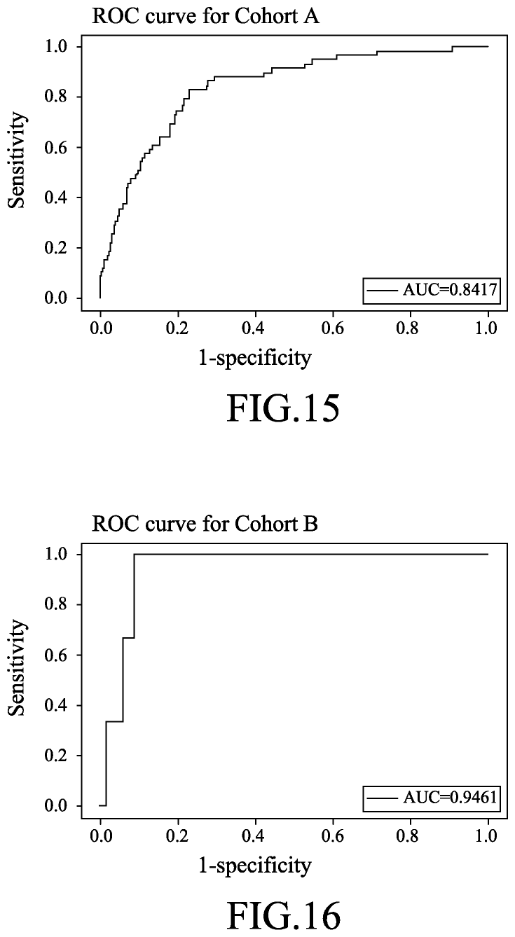

For the prediction model 22 according to the sixth embodiment, an ROC curve for the prediction model 22 fed with the validation data sets collected from Cohort A is illustrated in FIG. 15, and another ROC curve for the prediction model 22 fed with the validation data sets collected from Cohort B is illustrated in FIG. 16. An AUC related to the ROC curve in FIG. 15 is 0.8417, and an AUC related to said another ROC curve in FIG. 16 is 0.9461.

Referring to FIG. 2, in a seventh embodiment of the method, each of the plurality of training data sets includes the eGFR, the value of urine pH, the BMI and the age of a patient, the gender indicator indicating gender of the patient, the DM indicator indicating whether the patient was ever diagnosed with DM, the gout indicator indicating whether the patient was ever diagnosed with gout, the bacteriuria indicator indicating whether the patient was ever diagnosed with bacteriuria, and a hypertension indicator indicating whether the patient was ever diagnosed with hypertension. For example, the hypertension indicator may have a value of one to indicate that the patient had once been diagnosed with hypertension, or a value of zero to indicate that the patient was never diagnosed with hypertension. The prediction system 2 includes the eGFR computing unit 21.

In the seventh embodiment, the input variable set includes the eGFR, the value of urine pH, the BMI and the age of the subject, the gender indicator indicating gender of the subject, the DM indicator indicating whether the subject was ever diagnosed with DM, the gout indicator indicating whether the subject was ever diagnosed with gout, the bacteriuria indicator indicating whether the subject was ever diagnosed with bacteriuria, and a hypertension indicator indicating whether the subject was ever diagnosed with hypertension. For example, the hypertension indicator may have a value of one to indicate that the subject had once been diagnosed with hypertension, or a value of zero to indicate that the subject was never diagnosed with hypertension. Particularly, the prediction system 2 receives a variable x₀ that represents the creatinine concentration, a variable x₁ that represents the gender indicator, a variable x₂ that represents the age, a variable x₄ that represents the value of urine pH, a variable x₅ that represents the BMI, a variable x₆ that represents the DM indicator, a variable x₇ that represents the gout indicator, a variable x₈ that represents the bacteriuria indicator, and a variable x₉ that represents the hypertension indicator. The eGFR computing unit 21 calculates, based on the variables x₀,x₁ and x₂, a variable x₃ that represents the eGFR, and then the variable x₃ thus calculated is fed into the prediction model 22 together with the variables x₁,x₂,x₄,x₅,x₆,x₇,x₈ and x₉ as the input variable set for the prediction model 22 to obtain the probability of a kidney stone being a UA stone.

In the mathematical expression of the prediction model 22, ƒ(x) is a function that is mathematically expressed as ƒ(x)=w₀+w₁(x)ƒ₁(x)+w₂(x)ƒ₂(x)+w₃(x)ƒ₃(x)+w₄(x)ƒ₄(x)+w₅(x)ƒ₅(x)+w₆(x)ƒ₆(x), where w₀ is a constant, each of w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) is a function, and w₀, w₁(x), w₂(x), w₃(x), w₄(x), w₅(x), w₆(x), ƒ₁(x), ƒ₂(x), ƒ₃(x), ƒ₄(x), ƒ₅(x) and ƒ₆(x) are determined by using a fully connected neural network. In particular, the function ƒ(x) can be simplified as ƒ(x)=w₀+w₁ƒ₁(x₁)+w₂ƒ₂(x₂)+w₃ƒ₃(x₃)+w₄ƒ₄(x₄)+w₅ƒ₅(x₅)+w₆(x₉) ƒ₆(x₆,x₇,x₈), where each of w₀, w₁, w₂, w₃, w₄ and w₅ is a scalar trainable parameter, and each of ƒ₁(x₁), ƒ₂(x₂), ƒ₃(x₃), ƒ₄(x₄), ƒ₅(x₅), w₆(x₉) and ƒ₆(x₆,x₁,x₈) is a nonlinear function determined by using a fully connected two-layer neural network. For ƒ₁(x₁), a first layer of the fully connected two-layer neural network has a dimension m₁ that is an integer ranging from 1 to 100, and a second layer of the fully connected two-layer neural network has a dimension of 1. For ƒ₂(x₂), a first layer of the fully connected two-layer neural network has a dimension m₂ that is an integer ranging from 1 to 100, and a second layer of the fully connected two-layer neural network has a dimension of 1. For ƒ₃(x₃), a first layer of the fully connected two-layer neural network has a dimension m₃ that is an integer ranging from 1 to 100, and a second layer of the fully connected two-layer neural network has a dimension of 1. For ƒ₄(x₄), a first layer of the fully connected two-layer neural network has a dimension m₄ that is an integer ranging from 1 to 100, and a second layer of the fully connected two-layer neural network has a dimension of 1. For ƒ₅(x₅), a first layer of the fully connected two-layer neural network has a dimension m₅ that is an integer ranging from 1 to 100, and a second layer of the fully connected two-layer neural network has a dimension of 1. For ƒ₆(x₆,x₇,x₈), a first layer of the fully connected two-layer neural network has a dimension m₆ that is an integer ranging from 1 to 100, and a second layer of the fully connected two-layer neural network has a dimension of 1. For w₆(x₉), a first layer of the fully connected two-layer neural network has a dimension m₇ that is an integer ranging from 1 to 100, and a second layer of the fully connected two-layer neural network has a dimension of 1. Each of ƒ₁(x₁), ƒ₂(x₂), ƒ₃(x₃), ƒ₄(x₄) and ƒ₅(x₅) is mathematically expressed as

$${f}_{i} ( {x}_{i} ) = {σ}_{i , 2} ( {A}_{i , 2} {σ}_{i , 1} ( {A}_{i , 1} {x}_{i} + {B}_{i , 1} ) + {B}_{i , 2} ) ,$$

ƒ₆(x₆,x₇,x₈) is mathematically expressed as

$${f}_{6} ( {x}_{6} , {x}_{7} , {x}_{8} ) = {σ}_{6 , 2} ( {A}_{6 , 2} {σ}_{6 , 1} ( {A}_{6 , 1} {x}_{6} + {A}_{7 , 1} {x}_{7} + {A}_{8 , 1} {x}_{8} + {B}_{6 , 1} ) + {B}_{6 , 2} ) ,$$

and
w₆(x₉) is mathematically expressed as

$${w}_{6} ( {x}_{9} ) = {w}_{6 , 0} \cdot {σ}_{7 , 2} ( {A}_{7 , 2} {σ}_{7 , 1} ( {A}_{9 , 1} {x}_{9} + {B}_{7 , 1} ) + {B}_{7 , 2} ) ,$$

where i is equal to 1, 2, 3, 4, or 5, each of A_{i,1} and B_{i,1} is an mᵢ-by-1 vector containing mᵢ number of trainable model parameters, A_{i,2} is a 1-by-mᵢ vector containing m; number of trainable model parameters, Biz is a scalar trainable parameter, and each of σ_{i,1} and σ_{1,2} is a standard nonlinear activation function commonly used in building a neural network, each of A_{6,1}, A_{7,1}, A_{8,1} and B_{6,1} is a m₆-by-1 vector containing m₆ number of trainable model parameters, A_{6,2} is a 1-by-m₆ vector containing m₆ number of trainable model parameters, B_{6,2} is a scalar trainable parameter, each of σ_{6,1} and σ_{6,2} is a standard nonlinear activation function commonly used in building a neural network, each of A_{9,1}, and B_{7,1} is an m₇-by-1 vector containing my number of trainable model parameters, A_{7,2} is a 1-by-m₇ vector containing my number of trainable model parameters, each of B_{7,2} and w_{6,0} is a scalar trainable parameter, and each of σ_{7,1} and σ_{7,2} is a standard nonlinear activation function commonly used in building a neural network. In total, the function ƒ(x) contains Σᵢ₌₁⁷3mᵢ+2m₆+14 number of trainable model parameters. In this embodiment, each of m₁, m₂, m₃, m₄, m₅ and m₆ is equal to 20, m₇ is equal to 5, each of B_{1,1}, B_{2,1}, B_{3,1}, B_{4,1}, B_{5,1}, B_{6,1} and B_{7,1} is manually set as a zero vector, and the function ƒ(x) contains only 304 trainable model parameters, accordingly.

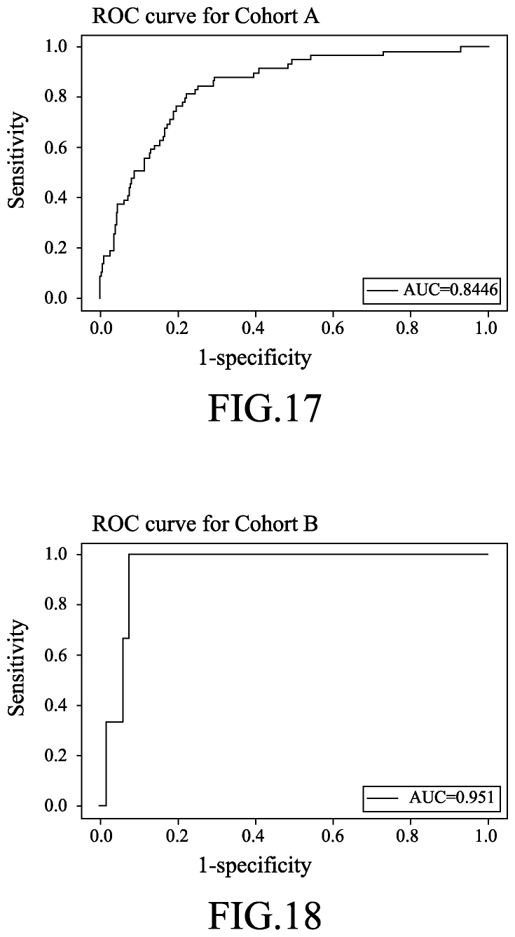

For the prediction model 22 according to the fifth embodiment, an ROC curve for the prediction model 22 fed with the validation data sets collected from Cohort A is illustrated in FIG. 17, and another ROC curve for the prediction model 22 fed with the validation data sets collected from Cohort B is illustrated in FIG. 18. An AUC related to the ROC curve in FIG. 17 is 0.8446, and an AUC related to said another ROC curve in FIG. 18 is 0.951.

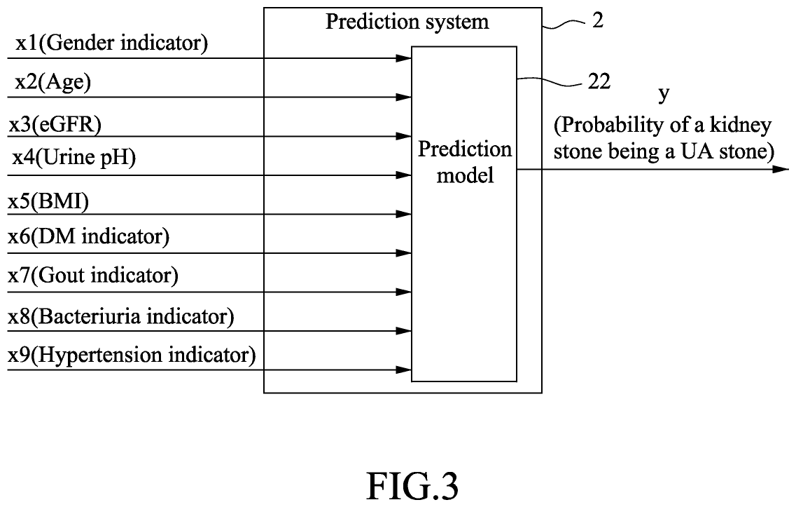

Referring to FIG. 3, an eighth embodiment of the method is similar to the seventh embodiment of the method, but is different therefrom in the following aspects. The prediction system 2 of the eighth embodiment does not include the eGFR computing unit 21 (see FIG. 2). The prediction system 2 receives a variable x₁ that represents the gender indicator, a variable x₂ that represents the age, a variable x₃ that represents the eGFR, a variable x₄ that represents the urine pH, a variable x₅ that represents the BMI, a variable x₆ that represents the DM indicator, a variable x₇ that represents the gout indicator, a variable x₈ that represents the bacteriuria indicator, and a variable x₉ that represents the hypertension indicator. The prediction system 2 directly feeds the variables x₁,x₂,x₃,x₄,x₅,x₆,x₇,x₈ and x₉ thus received into the prediction model 22 as the input variable set for the prediction model 22 to obtain the probability of a kidney stone being a UA stone.

In one embodiment, the prediction model is mathematically expressed as

$$y = \frac{1}{1 + {e}^{f ( x )}} ,$$

where y represents output of the prediction model 22, x represents input to the prediction model 22, and ƒ(x) is a function that is mathematically expressed as

$$f ( x ) = {w}_{0} + {w}_{1} ( x ) {f}_{1} ( x ) + … + {w}_{i} ( x ) {f}_{i} ( x ) + … + {w}_{n} ( x ) {f}_{n} ( x ) ,$$

where n is a positive integer greater than one, i is a positive integer ranging between one and n, w₀ is a constant, each of w₁(x), wᵢ(x), wₙ(x), ƒ₁(x), ƒᵢ(x) and ƒₙ(x) is a function, and w₀, w₁(x), wᵢ(x), wₙ(x), ƒ₁(x), ƒᵢ(x) and ƒₙ(x) are determined by using a fully connected neural network.

To sum up, for the method for determining a probability of a kidney stone in a subject being a UA stone according to the disclosure, a machine-learning-based prediction model is established and is utilized to obtain the probability based on an input variable set that is related to the subject and that includes at least an eGFR and a value of urine pH, which are easily available at an initial clinical visit of the subject. It is worth to note that results of validation exemplarily shown by AUCs of ROC curves illustrated in FIGS. 5 to 18 indicate that the probability obtained by using the method according to the disclosure is highly accurate. Thus, a medical practitioner is able to conveniently and reliably determine whether a kidney stone in a subject is a UA stone, and to provide timely and appropriate treatments for the subject.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for determining a probability of a kidney stone in a subject being a uric-acid (UA) stone, comprising steps of:
establishing, by using a machine learning algorithm, a prediction model based on a plurality of training data sets that are related to a plurality of patients, each of the plurality of training data sets at least including an estimated glomerular filtration rate (eGFR) and a value of urine pH; and
feeding an input variable set into the prediction model so as to obtain the probability of the kidney stone in the subject being a UA stone, the input variable set being related to the subject and including an eGFR and a value of urine pH of the subject.

2. The method as claimed in claim 1, wherein:
the prediction model is mathematically expressed
$$y = \frac{1}{1 + {e}^{f ( x )}} ,$$
where y represents output of the prediction model, x represents input to the prediction model, and ƒ(x) is a function that is mathematically expressed as
ƒ(x)=w₀+w₃(x)ƒ₃(x)+w₄(x)ƒ₄(x), where w₀ is a constant, each of w₃(x), w₄(x), ƒ₃(x) and ƒ₄(x) is a function, and w₀, w₃(x), w₄(x), ƒ₃(x) and ƒ₄(x) are determined by using a fully connected neural network.

3. The method as claimed in claim 1, wherein:
the prediction model is mathematically expressed
$$y = \frac{1}{1 + {e}^{f ( x )}} ,$$
where y represents output of the prediction model, x represents input to the prediction model, and ƒ(x) is a function that is mathematically expressed as
ƒ(x)=w₀+w₃(x)ƒ₃(x)+w₄(x)ƒ₄(x)+w₅(x)ƒ₅(x), where w₀ is a constant, each of w₃(x), w₄(x), w₅(x), ƒ₃(x), ƒ₄(x) and ƒ₅(x) is a function, and w₀, w₃(x), w₄(x), w₅(x), ƒ₃(x), ƒ₄(x) and ƒ₅(x) are determined by using a fully connected neural network.

4. The method as claimed in claim 1, wherein:
the prediction model is mathematically expressed
$$y = \frac{1}{1 + {e}^{f ( x )}} ,$$
where y represents output of the prediction model, x represents input to the prediction model, and ƒ(x) is a function that is mathematically expressed as
$$f ( x ) = {w}_{0} + {w}_{2} ( x ) {f}_{2} ( x ) + {w}_{3} ( x ) {f}_{3} ( x ) + {w}_{4} ( x ) {f}_{4} ( x ) + {w}_{5} ( x ) {f}_{5} ( x ) ,$$
where w₀ is a constant, each of w₂(x), w₃(x), w₄(x), w₅(x), ƒ₂(x), ƒ₃(x), ƒ₄(x) and ƒ₅(x) is a function, and w₀, w₂(x), w₃(x), w₄(x), w₅(x), ƒ₂(x), ƒ₃(x), ƒ₄(x) and ƒ₅(x) are determined by using a fully connected neural network.

5. The method as claimed in claim 1, wherein:
the prediction model is mathematically expressed
$$y = \frac{1}{1 + {e}^{f ( x )}} ,$$
where y represents output of the prediction model, x represents input to the prediction model, and ƒ(x) is a function that is mathematically expressed as
ƒ(x)=w₀+w₁(x)ƒ₁(x)+ . . . +wᵢ(x)ƒᵢ(x)+ . . . +wₙ(x)ƒₙ(x), where n is a positive integer greater than one, i is a positive integer ranging between one and n, w₀ is a constant, each of w₁(x), wᵢ(x), wₙ(x), ƒ₁(x), ƒᵢ(x) and ƒₙ(x) is a function, and w₀, w₁(x), wᵢ(x), wₙ(x), ƒ₁(x), ƒᵢ(x) and ƒₙ(x) are determined by using a fully connected neural network.

6. The method as claimed in claim 5, wherein n is equal to five.

7. The method as claimed in claim 5, wherein n is equal to six.

8. The method as claimed in claim 1, wherein the input variable set further includes a body mass index (BMI) that is related to the subject.

9. The method as claimed in claim 1, wherein the input variable set further includes an age of the subject.

10. The method as claimed in claim 1, wherein the input variable set further includes a gender indicator that indicates gender of the subject.

11. The method as claimed in claim 1, wherein the input variable set further includes a diabetes mellitus (DM) indicator that indicates whether the subject was ever diagnosed with DM.

12. The method as claimed in claim 1, wherein the input variable set further includes a gout indicator that indicates whether the subject was ever diagnosed with gout, and a bacteriuria indicator that indicates whether the subject was ever diagnosed with bacteriuria.

13. The method as claimed in claim 1, wherein the input variable set further includes a hypertension indicator that indicates whether the subject was ever diagnosed with hypertension.

14. The method as claimed in claim 1, wherein the input variable set further includes an age of the subject, a gender indicator that indicates gender of the subject, and a creatinine concentration that is related to the subject, and wherein prior to feeding an input variable set into a prediction model, the method further comprises a step of:
determining the eGFR of the input variable set based on the age, the gender indicator and the creatinine concentration.

15. The method as claimed in claim 14, wherein the step of determining the eGFR is to calculate the eGFR by using the isotope dilution mass spectrometry traceable Modification of Diet in Renal Disease formula that is mathematically expressed as:
$$eGFR [ mL / min / 1.73 {m}^{2} ] = 175 \times {( Scr )}^{- 1.154} \times {( Age )}^{- 0.203} \times {( 0.742 )}^{G} ,$$
where Scr represents the creatinine concentration, Age represents the age, and G represents the gender, has a value of one when the gender indicator indicates that the subject is female, and has a value of zero when the gender indicator indicates that the subject is male.

16. The method as claimed in claim 1, wherein:
the prediction model is mathematically expressed
$$y = \frac{1}{1 + {e}^{f ( x )}} ,$$
where y represents output of the prediction model, x represents input to the prediction model, and ƒ(x) is a function and includes one of w₀, w₁(x)ƒ₁(x), . . . , wᵢ(x)ƒᵢ(x), . . . , wₙ(x)ƒₙ(x) and any combination thereof, where n is a positive integer greater than one, i is a positive integer ranging between one and n, w₀ is a constant, each of w₁(x), wᵢ(x), wₙ(x), ƒ₁(x), ƒᵢ(x) and ƒₙ(x) is a function, and w₀, w₁(x), wᵢ(x), wₙ(x), ƒ₁(x), ƒᵢ(x) and ƒₙ(x) are determined by using a fully connected neural network.
